(19) United States
(12) Patent Application Publication
(10) Pub. No.: US 2009/0060847 A1
(43) Pub. Date: Mar. 5, 2009

(54) CONTRAST AGENT FOR ULTRASOUND EXAMINATION OF THE PROSTATE AND METHOD TO DIAGNOSE PROSTATE CANCER

(76) Inventors: Sueleyman Erguen, Essen (DE); Jens Fehre, Hausen (DE); Ralf Nanke, Neunkirchen am Brand (DE); Bernhard Singer, Essen (DE); Derya Tilki, Muenchen (DE)

Correspondence Address: SCHIFF HARDIN, LLP;PATENT DEPARTMENT, 6600 SEARS TOWER, CHICAGO, IL, 60606-6473, US

(21) Appl. No.: 12/203,193

(22) Filed: Sep. 3, 2008

(30) Foreign Application Priority Data
Sep. 3, 2007 (DE) 10 2007 041 831.2

(51) Int. Cl. A61B 8/08

(52) U.S. Cl. 424/9.52; 424/9.5

(57) ABSTRACT

A contrast agent for ultrasound examination of the prostate has contrast-intensifying particles on an exterior of which at least one binding molecule is present that specifically binds to CEACAM-1. In a method for ultrasound examination of the prostate, such a contrast agent is administered to the patient and a sonography of the prostate is subsequently implemented.

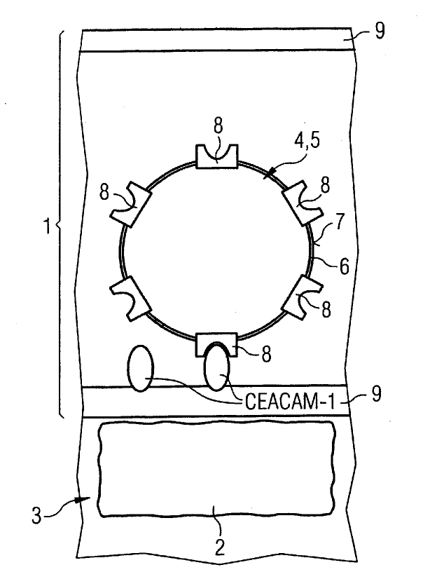

### BACKGROUND OF THE INVENTION

1. Field of the Invention

The invention concerns a contrast agent for ultrasound examination as well as a method to diagnose prostate cancer.

2. Description of the Prior Art

Today, in addition to digital rectal examination (palpation) and the determination of the prostate-specific antigen (PSA blood test), transrectal ultrasound examination (TRUS=transrectal ultrasonography) is one of the three pillars of present prostate cancer diagnosis in everyday clinical life. However, like the other standard methods, ultrasound examination exhibits an insufficient sensitivity and specificity. The cause of this is, among other things, that prostate cancers can be both echopenic, or equally echogenic as healthy tissue or more echogenic than healthy tissue. Ultrasound sonography is used primarily in the control of biopsies in the diagnosis of prostate cancer. Given a conspicuous preliminary finding, multiple punch cylinders are extracted from the prostate under ultrasound monitoring, which punch cylinders are subsequently histologically examined. Since the cancers can often not be seen in the ultrasound image, essentially a blind biopsy occurs, i.e. the samples are taken not from suspected areas but from all regions of the prostate according to a predetermined plan. The application of ultrasound thus is used only for anatomical orientation. The success of the biopsy thus depends on the hit probability, which is why many small tumors remain undetected in the early stage, such that multiple re-biopsies are required.

The new formation of blood vessels (angiogenesis/vasculogenesis) plays an important role for the growth of tumors which can also be utilized for ultrasound imaging. The intensified blood flow in the small blood vessels can be shown with the use of Doppler examinations and the use of what are known as micro-bubbles. Possible tumors or suspect areas for the diagnose and the biopsy thus can be better identified than in conventional B-image representation. The micro-bubbles (a few μm in size on average) consist of a shell (for example made of lipids) and are filled with air or gas, which results in a strong contrast in the ultrasound image (directly or by excitation).

Moreover, pre-clinical studies with regard to the use of what are known as targeted micro-bubbles are presently running. These are thereby micro-bubbles on whose shell coupled molecules (for example antibodies) are attached that should dock with specific target molecules in the body. The goal is for the micro-bubbles to accumulate in very specific target areas so as to enable an ultrasound-assisted imaging of this area. Prostate-nonspecific target molecules indicating angiogenesis (such as, for example, VEGF-R2 (KDR) or alpha(v)-beta(3)-integrin) are examined as possible target molecules.

### SUMMARY OF THE INVENTION

An object of the invention is to provide a contrast agent for ultrasound examination of the prostate and a method to diagnose prostate cancer with which a prostate tumor can already be detected or treated in the early stage.

This object is achieved by a contrast agent according to the invention that contains contrast agent-intensified particles on the surface of which at least one binding molecule is present that specifically binds to CEACAM-1. The invention is based on research results from Tilki et al. (Oncogene (2006) 25, 4965-4974), according to which CEACAM-1 occurs only in the epithelium of a healthy prostate but not in the blood vessels of the prostate. In an early phase of tumor development (for instance in the stage of hgPIN (high grade epithelial prostatic neoplasia) in which the tumor has not yet formed its own vessels), the expression of CEACAM-1 in the prostate epithelium is regulated down while it is regulated up in the endothelium of adjoining small blood vessels of healthy tissue. According to the invention, this effect is now utilized to detect an early cancer stage. In contrast to a healthy prostate, a completely different accumulation [uptake; enrichment] image results given the presence of a tumor in the early stage. A concentration of the contrast agent in the region of the tumor occurs here, such that this is visible in the ultrasound image.

The above statements apply analogously to the method according to the invention to diagnose prostate cancer, in which method a contrast agent as described above is administered to the patient and sonography of the prostate is subsequently implemented.

### BRIEF DESCRIPTION OF THE DRAWING

The single FIGURE schematically illustrates a region of a prostate showing a blood vessel and a tumor therein.

### DESCRIPTION OF THE PREFERRED EMBODIMENTS

The FIGURE is a schematic representation of a region of the prostate 3 having a blood vessel 1 and a tumor 2. The FIGURE shows the structure of the contrast agent and enrichment thereof in the blood vessels 1. A contrast agent that can be supplied to the prostate 3 via the circulatory system (for instance after intravenous administration, for example) contains particles 4 that produce an ultrasound echo in sonography, which is more pronounced than the echo of a tissue region of the prostate 3 or tumor 2. The contrast agent has bubbles a few micrometers in size, filled with air or another gas, with a shell of lipids, for example. In addition to micro-bubbles, echogenic substances can also be used, for example particles formed from biopolymers. Alignate and chitin are in particular to be cited as suitable materials.

Binding molecules 8 that specifically bind to CEACAM-1 molecules (carcinoembryonic antigen-related cell adhesion molecules) can be indirectly or directly connected to the outside 7 of the shell. These molecules form in blood vessels 1 that are adjacent to the tumor tissue. This thereby leads to an enrichment of contrast agent particles in the tissue of the prostate 3 that is adjacent to the tumor 2. The CEACAM-1 molecules are arranged in the wall 9 (and in fact in its endothelium) and therefore can be reached via the circulatory system. CEACAM-1 antibodies primarily serve as binding molecules 8. However, genetically engineered soluble forms of CEACAM-1 are also suitable. Aptamers, spiegelmers and/or anticalins are likewise suitable. Aptamers are short, stable and specifically binding RNA chains, spiegelmers are their mirror-image counterparts. The anticalins are individual polypeptide chains with approximately 180 amino acids that possess specific binding properties similar to those of antibodies but are easier to produce than these.

An ultrasound examination can proceed, for example, as follows: The contrast agent is injected into the patient. The prostate is monitored with the use of ultrasound sonography, with a rectal probe being appropriately used that carries, in a known manner, an ultrasound transmitter and receiver. The contrast agent has distributed in the body after a certain amount of time has elapsed, so an enrichment occurs in tissue regions having a vascular structure in which CEACAM-1 is present. The enrichment occurs due to the binding between a binding molecule 8 and CEACAM-1. Given the presence of a tumor in the early stage, contrast agent enrichment thereof is observed in the region of the tumor (i.e. in its adjacent small vessels). The contrast agent particles 4 are more echogenic than the surrounding tissue. Relatively small tumor foci that cannot be detected with conventional methods thereby become visible.

Although modifications and changes may be suggested by those skilled in the art, it is the intention of the inventors to embody within the patent warranted hereon all changes and modifications as reasonably and properly come within the scope of their contribution to the art.

We claim as our invention:

1. A contrast agent for ultrasound examination of the prostate, comprising contrast-intensifying particles having an exterior at which at least one binding molecule is present that specifically binds to CEACAM-1.

2. A contrast agent according to claim 1, wherein the contrast-intensifying particles are formed of micro-bubbles.

3. A contrast agent according to claim 1, wherein the contrast-intensifying particles are formed of biopolymers.

4. A contrast agent according to claim 3, wherein at least some of the particles consist of chitin.

5. A contrast agent according to claim 3, wherein at least some of the particles consist of alginate.

6. A contrast agent according to claim 1, wherein CEACAM-1 antibodies are present as binding molecules.

7. A contrast agent according to claim 1, wherein genetically engineered, soluble forms of CEACAM-1 are present as binding molecules.

8. A contrast agent according to claim 1, wherein aptamers, spiegelmers and/or anticalins are present as binding molecules.

9. A method for diagnosing prostate cancer, comprising the steps of:
administering a contrast agent to a patient comprising contrast-intensifying particles having an exterior at which at least one binding molecule is present that specifically binds to CEACAM-1; and
implementing sonography of the prostate of the patient enhanced with said contrast agent.

10. A method as claimed in claim 9 comprising implementing said sonography of the prostate rectally.
